(19) United States
(12) Patent Application Publication
(10) Pub. No.: US 2006/0184166 A1
(43) Pub. Date: Aug. 17, 2006

(54) Method and apparatus to automatically insert a probe into a cornea

(76) Inventors: Moises Valle, Tustin, CA (US); Steve Khalaj, Laguna Hills, CA (US); Larry Hood, Laguna Hills, CA (US); Dorin Panescu, San Jose, CA (US)

Correspondence Address: IRELL & MANELLA LLP, 840 NEWPORT CENTER DRIVE, SUITE 400, NEWPORT BEACH, CA, 92660, US

(21) Appl. No.: 11/060,152

(22) Filed: Feb. 16, 2005

(51) Int. Cl. A61B 18/14

(52) U.S. Cl. 606/41; 606/45; 606/48

(57) ABSTRACT

An apparatus that is used to perform a medical procedure on a cornea. The apparatus may include a ring that can be placed on a cornea and a probe that can deliver energy to denature corneal tissue. The probe can be moved about the ring and cornea by a first actuator. A second actuator may move the probe into contact with the cornea to deliver energy and denature tissue. The process of moving the probe and delivering energy can be repeated to create a circular pattern of denatured areas. The circular pattern of denatured areas may correct for hyperopia. The actuators may be controlled by a controller that operates in accordance with a program to move the probe and create the circular pattern of denatured areas in an automated process.

### BACKGROUND OF THE INVENTION

1. Field of the Invention

The present invention relates to a thermokeratoplasty system that is used to reshape a cornea.

2. Prior Art

Techniques for correcting vision have included reshaping the cornea of the eye. For example, myopic conditions can be corrected by cutting a number of small incisions in the corneal membrane. The incisions allow the corneal membrane to relax and increase the radius of the cornea. The incisions are typically created with either a laser or a precision knife. The procedure for creating incisions to correct myopic defects is commonly referred to as radial keratotomy and is well known in the art.

Radial keratotomy techniques generally make incisions that penetrate approximately 95% of the cornea. Penetrating the cornea to such a depth increases the risk of puncturing the Descemets membrane and the endothelium layer, and creating permanent damage to the eye. Additionally, light entering the cornea at the incision sight is refracted by the incision scar and produces a glaring effect in the visual field. The glare effect of the scar produces impaired night vision for the patient.

The techniques of radial keratotomy are only effective in correcting myopia. Radial keratotomy cannot be used to correct an eye condition such as hyperopia. Additionally, radial keratotomy has limited use in reducing or correcting an astigmatism. The cornea of a patient with hyperopia is relatively flat (large spherical radius). A flat cornea creates a lens system which does not correctly focus the viewed image onto the retina of the eye. Hyperopia can be corrected by reshaping the eye to decrease the spherical radius of the cornea. It has been found that hyperopia can be corrected by heating and denaturing local regions of the cornea. The denatured tissue contracts and changes the shape of the cornea and corrects the optical characteristics of the eye. The procedure of heating the corneal to correct a patient's vision is commonly referred to as thermokeratoplasty.

U.S. Pat. No. 4,461,294 issued to Baron; U.S. Pat. No. 4,976,709 issued to Sand and PCT Publication WO 90/12618, all disclose thermokeratoplasty techniques which utilize a laser to heat the cornea. The energy of the laser generates localized heat within the corneal stroma through photonic absorption. The heated areas of the stroma then shrink to change the shape of the eye.

Although effective in reshaping the eye, the laser based systems of the Baron, Sand and PCT references are relatively expensive to produce, have a non-uniform thermal conduction profile, are not self limiting, are susceptible to providing too much heat to the eye, may induce astigmatism and produce excessive adjacent tissue damage, and require long term stabilization of the eye. Expensive laser systems increase the cost of the procedure and are economically impractical to gain widespread market acceptance and use.

Additionally, laser thermokeratoplasty techniques non-uniformly shrink the stroma without shrinking the Bowmans layer. Shrinking the stroma without a corresponding shrinkage of the Bowmans layer, creates a mechanical strain in the cornea. The mechanical strain may produce an undesirable reshaping of the cornea and probable regression of the visual acuity correction as the corneal lesion heals. Laser techniques may also perforate Bowmans layer and leave a leucoma within the visual field of the eye.

U.S. Pat. Nos. 4,326,529 and 4,381,007 issued to Doss et al, disclose electrodes that are used to heat large areas of the cornea to correct for myopia. The electrode is located within a sleeve that suspends the electrode tip from the surface of the eye. An isotropic saline solution is irrigated through the electrode and aspirated through a channel formed between the outer surface of the electrode and the inner surface of the sleeve. The saline solution provides an electrically conductive medium between the electrode and the corneal membrane. The current from the electrode heats the outer layers of the cornea. Heating the outer eye tissue causes the cornea to shrink into a new radial shape. The saline solution also functions as a coolant which cools the outer epithelium layer.

The saline solution of the Doss device spreads the current of the electrode over a relatively large area of the cornea. Consequently, thermokeratoplasty techniques using the Doss device are limited to reshaped corneas with relatively large and undesirable denatured areas within the visual axis of the eye. The electrode device of the Doss system is also relatively complex and cumbersome to use.

“A Technique for the Selective Heating of Corneal Stroma” Doss et al., Contact & Intraoccular Lens Medical Jrl., Vol. 6, No. 1, pp. 13-17, January-March, 1980, discusses a procedure wherein the circulating saline electrode (CSE) of the Doss patent was used to heat a pig cornea. The electrode provided 30 volts r.m.s. for 4 seconds. The results showed that the stroma was heated to 70° C. and the Bowman's membrane was heated 45° C., a temperature below the 50-55° C. required to shrink the cornea without regression.

“The Need For Prompt Prospective Investigation” McDonnell, Refractive & Corneal Surgery, Vol. 5, January/February, 1989 discusses the merits of corneal reshaping by thermokeratoplasty techniques. The article discusses a procedure wherein a stromal collagen was heated by radio frequency waves to correct for a keratoconus condition. As the article reports, the patient had an initial profound flattening of the eye followed by significant regression within weeks of the procedure.

“Regression of Effect Following Radial Thermokeratoplasty in Humans” Feldman et al., Refractive and Corneal Surgery, Vol. 5, September/October, 1989, discusses another thermokeratoplasty technique for correcting hyperopia. Feldman inserted a probe into four different locations of the cornea. The probe was heated to 600° C. and was inserted into the cornea for 0.3 seconds. Like the procedure discussed in the McDonnell article, the Feldman technique initially reduced hyperopia, but the patients had a significant regression within 9 months of the procedure.

Refractec, Inc. of Irvine Calif., the assignee of the present application, has developed a system to correct hyperopia and presbyopia with a thermokeratoplasty probe that is connected to a console. The probe includes a tip that is inserted into the stroma layer of a cornea. Radio frequency (“RF”) electrical current provided by the console flows through the eye to denature the collagen tissue within the stroma. The process of inserting the probe tip and applying electrical current can be repeated in a circular pattern about the cornea. The procedure of applying RF electrical energy through a probe tip to denature corneal tissue is taught by Refractec under the service marks CONDUCTIVE KERATOPLASTY and CK.

In a CK procedure, probe tip placement is initially marked with a corneal marker. The doctor must then manually push the probe tip into the marked locations to deliver RF energy. Manual placement and insertion of the tip allows for human error. It would be desirable to provide a system that can automatically locate the probe on the cornea to minimize human error in a CK procedure.

### BRIEF SUMMARY OF THE INVENTION

An apparatus that is used to perform a medical procedure on a cornea. The apparatus includes a probe that delivers energy and a mechanism that can move the probe about the cornea, and into contact with the cornea.

### BRIEF DESCRIPTION OF THE DRAWINGS

FIG. 1 is a perspective view of a thermokeratoplasty system;

FIG. 2 is a perspective view of a ring assembly of the system;

FIG. 3 is a side section view of the ring assembly;

FIG. 4 is a schematic of a controller;

FIG. 5 is a graph showing a waveform that is provided by a controller of the system;

FIG. 6 is an illustration showing a pattern of denatured areas of a cornea.

### DETAILED DESCRIPTION

Disclosed is an apparatus that is used to perform a medical procedure on a cornea. The apparatus may include a ring that can be placed on a cornea and a probe that can deliver energy to denature corneal tissue. The probe can be moved about the ring and cornea by a first actuator. A second actuator may move the probe into contact with the cornea to deliver energy and denature tissue. The process of moving the probe and delivering energy can be repeated to create a circular pattern of denatured areas. The circular pattern of denatured areas may correct for hyperopia. The actuators may be controlled by a controller that operates in accordance with a program to move the probe and create the circular pattern of denatured areas in an automated process.

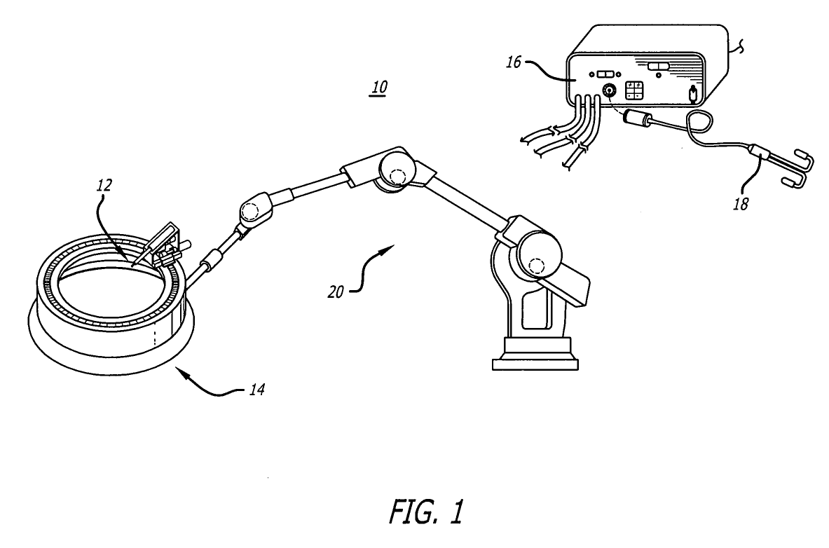

Referring to the drawings more particularly by reference numbers, FIG. 1 shows a system 10 that can be used to perform a medical procedure on a cornea. The system 10 includes a probe 12 coupled to an automated suction ring assembly 14. The suction ring assembly 14 can move the probe 12. The probe 12 and ring assembly 14 are coupled to a controller 16. The controller 16 can provide energy that is delivered by the probe 12. The controller 16 can also control the ring assembly 14 to move the probe 12 to different locations on a cornea, and to move the probe 12 into contact with the cornea.

The probe 12 may be a mono-polar or a bipolar electrode device. If the probe is mono-polar the system 10 may also have a return element 18 that is in contact with the patient to provide a return path for the electrical current provided by the controller 16 to the probe 12. By way of example, the return element 18 may be integral with a lid speculum that is used to maintain the patient's eyelids in an open position while a procedure is performed.

The ring assembly 12 may be moved through an arm 20. The arm 20 may have a plurality of joints to provide multiple degrees of freedom. The arm 20 may have counterweights and/or springs that can balance and maintain the position of the ring assembly 12 on a cornea to minimize patient discomfort.

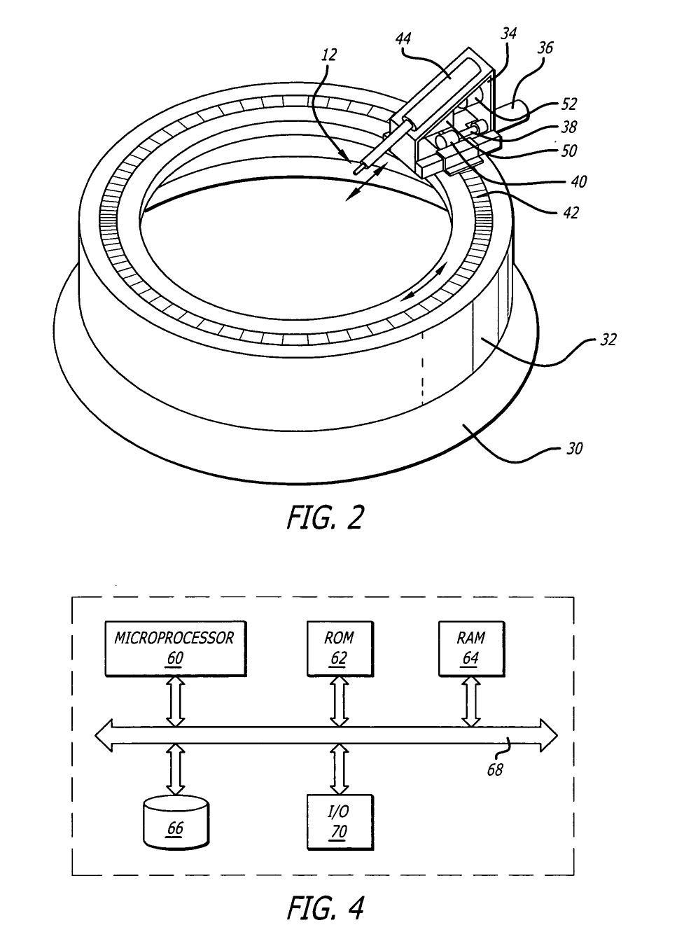

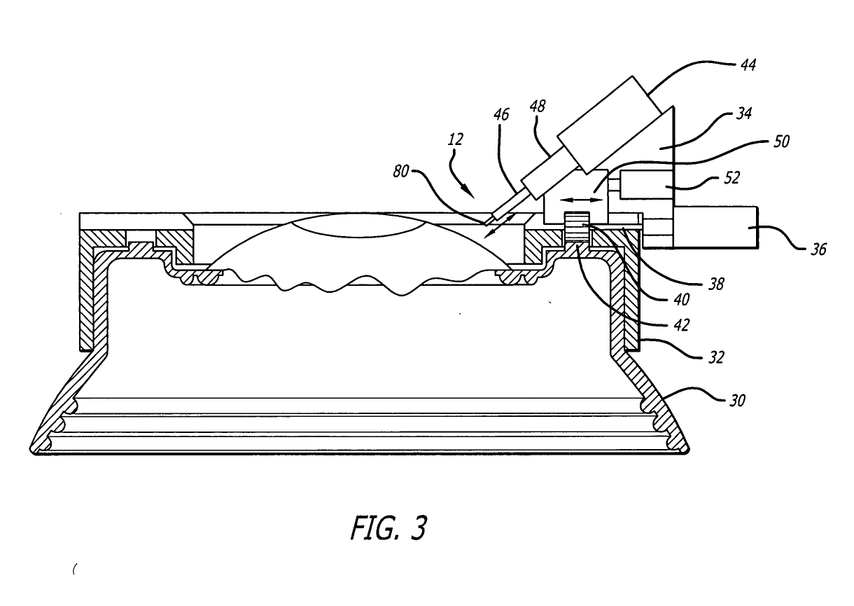

FIGS. 2 and 3 show an embodiment of a ring assembly 12. The ring assembly 12 may include a suction ring 30 that can be placed onto a cornea. Although a circular ring is shown it is to be understood that other geometries may be employed. The suction ring 30 may contain apertures, channels, etc. that are coupled to a source of vacuum. The vacuum source creates a vacuum pressure that maintains the position of the ring 30 on the cornea.

The ring assembly 12 may further contain a sliding collar 32 that can rotate about the suction ring 30 in either a clockwise or counterclockwise direction as indicated by the arrows. A block 34 may be attached to the sliding collar 32. The block 34 supports a first actuator 36 that can move the sliding collar 32 around the ring 30. The first actuator 36 may include a rotating output shaft 38 that has a pinion gear 40. The top surface of the suction ring 30 may have mating gear teeth 42 to form a rack and pinion gear assembly. Rotation of the output shaft 38 causes the sliding collar 32 to move about the ring 30.

The first actuator 36 may be an electrical motor that receives input signals from the controller 16. The controller 16 can activate and de-active the motor to control the movement of the sliding collar 32 and the position of the probe 12.

The ring assembly 14 may further have a second actuator 44 that is supported by the block 34 and attached to the probe 12. The second actuator 44 can move the probe 12 in a linear matter as indicated by the arrows. The second actuator 44 can move the probe 12 into and out of contact with the cornea. The second actuator 44 may also be an electric motor that is controlled by the controller 16.

The second actuator 44 may have an output shaft 46 that is attached to the probe 12 and can slide through an outer sleeve 48. The sleeve 48 is attached to the motor housing and is in contact with a sliding block 50. The sliding block 50 is moved in a linear manner by a third actuator 52 as indicated by the arrows. The third actuator 52 is attached to the outer block 34. Actuation of the third actuator 52 slides the block and moves the probe 12 to different radius positions of the cornea. By way of example, the probe 12 can be moved between 2 to 5 millimeters from the center of a cornea.

As shown in FIG. 4 the controller 16 may include at least one microprocessor 60, volatile memory (RAM) 62, non-volatile memory (ROM) 64 and a mass storage device (HDD) 66 all connected to a bus 68. The controller 16 may have I/O ports 70 with associated drivers, A/D, D/A, etc. circuits for interfacing with the probe 12 and ring assembly 14.

The processor 60 may perform operations in accordance with data and instructions provided by software/firmware. By way of example, the processor 60 may operate in accordance with a program that causes actuation of the second actuator 44 to move the probe 12 into contact with a cornea, delivery energy through the probe 12 to denature corneal tissue, and then activate the second actuator 44 to move the probe 12 out of contact with the cornea. The controller 16 may then activate the first actuator 36 to move the probe 12 to a new location wherein the process of activating the second actuator 44, delivering energy, and de-activating the second actuator 44 is repeated to create a second denatured spot. The process can repeated to create a desired pattern of denatured spots. The controller 16 can also activate the third actuator 52 to move the radius position of the probe 12 relative to the cornea.

The controller 16 may provide a predetermined amount of energy, through a controlled application of power for a predetermined time duration. The controller 16 may have manual controls that allow the user to select treatment parameters such as the power and time duration. The controller may have monitors and feedback systems for measuring physiologic tissue parameters such as tissue impedance, tissue temperature, tissue opacity and other parameters, and adjust the output power of the radio frequency generator to accomplish the desired results.

In one embodiment, actuators 36 and 44 work in an open-loop configuration that requires user interaction to return to a home, or reference, position such that accurate denatured spot placement is achieved. In another embodiment, actuators 36 and 44 work in a closed-loop configuration where information for positioning sensors, such as encoders, is provided to control the return of these actuators to a home, or reference, position and then precise locations where creation of denatured spots is desired.

In one embodiment, the controller 16 provides voltage limiting to prevent arcing. To protect the patient from overvoltage or overpower, the controller 16 may have an upper voltage limit and/or upper power limit which terminates power to the probe when the output voltage or power of the unit exceeds a predetermined value.

The controller 16 may also contain sensor and alarm circuits which monitor physiologic tissue parameters such as the resistance or impedance of the load or other measurable parameters, and provides adjustments and/or an alarm when the resistance/impedance value or other parameter exceeds and/or falls below predefined limits. The adjustment feature may change the voltage, current, and/or power delivered by the console such that the physiological parameter is maintained within a certain range. The alarm may provide either an audio and/or visual indication to the user that the resistance/impedance value has exceeded the outer predefined limits. Additionally, the unit may contain a ground fault indicator, and/or a tissue temperature sensor. The front panel of the controller 16 typically contains indicators and displays that provide an indication of the power, frequency, etc., of the power delivered to the probe.

The controller 16 may deliver a radiofrequency (RF) electrical power output in a frequency range of 50 KHz-30 MHz. In the preferred embodiment, power is provided to the probe at a frequency in the range of 350 KHz. The controller 16 is designed so that the power supplied to the probe 12 does not exceed a certain upper limit of up to several watts. Preferably the console is set to have an upper power limit of 1.2 watts (W). The time duration of each application of power to a particular corneal location can be up to several seconds but is typically set between 0.1-1.0 seconds. The unit 16 is preferably set to deliver approximately 0.6 W of power for 0.6 seconds.

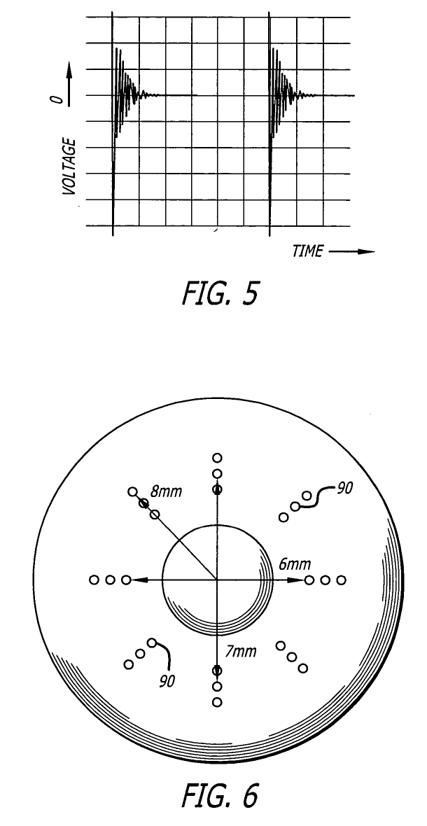

FIG. 5 shows a typical voltage waveform that is delivered by the probe 12 to the cornea. Each pulse of energy delivered by the probe 12 may be a highly damped sinusoidal waveform, typically having a crest factor (peak voltage/RMS voltage) greater than 5:1. Each highly damped sinusoidal waveform is repeated at a repetition rate. The repetition rate may vary between 1-40 KHz and is preferably set at 7.5 KHz. Although a damped waveform is shown and described, other waveforms, such as continuous sinusoidal, amplitude, frequency or phase-modulated sinusoidal, pulsed, pulse width modulated etc. can be employed.

The probe 12 provides a current to the cornea through a probe tip 80 (see FIG. 3). The current denatures the collagen tissue of the stroma. The tip 30 typically is preferably inserted into the stroma layer of a cornea. Because the tip 80 is inserted into the stroma it has been found that a power no greater than 1.2 watts for a time duration no greater than 1.0 seconds will adequately denature the corneal tissue to provide optical correction of the eye. However, other power and time limits, in the range of several watts and seconds, respectively, can be used to effectively denature the corneal tissue. Inserting the tip 80 into the cornea provides improved repeatability over probes placed into contact with the surface of the cornea, by reducing the variances in the electrical characteristics of the epithelium and the outer surface of the cornea.

FIG. 6 shows a pattern of denatured areas 90 that have been found to correct hyperopic or presbyopic conditions. A circle of 8, 16, or 24 denatured areas 90 are created about the center of the cornea, outside the visual axis portion of the eye. The visual axis has a nominal diameter of approximately 5 millimeters. It has been found that 16 denatured areas provide the most corneal shrinkage and less post-op astigmatism effects from the procedure. The circle of denatured areas typically have a diameter between 6-8 mm, with a preferred diameter of approximately 7 mm. If the first circle does not correct the eye deficiency, the same pattern may be repeated, or another pattern of 8 denatured areas may be created within a circle having a diameter of approximately 6.0-6.5 mm either in line or overlapping. The diameter of the circular pattern(s) can be established by activation of the third actuator by the controller 16. Refractec, Inc. provides instructional services to educate those performing such procedures under the service marks CONDUCTIVE KERATOPLASTY and CK. The pattern of denatured areas can be programmed into the controller 16.

The exact diameter of the pattern may vary from patient to patient, it being understood that the denatured spots should preferably be formed outside the visual axis of the eye. Although a circular pattern is shown, it is to be understood that the denatured areas 90 may be located in any location and in any pattern. In addition to correcting for hyperopia, the present invention may be used to correct astigmatic or other visual conditions. For correcting astigmatic conditions, the denatured areas are typically created at the end of the astigmatic flat axis. The present invention may also be used to correct procedures that have overcorrected for a myopic condition.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art. Although this disclosure describes a ring-shaped mechanism for actuators, other geometries can be employed (square, toroidal, etc.) without departing from the spirit of the invention.

For example, although the delivery of radio frequency energy is described, it is to be understood that other types of non-thermal energy such as direct current (DC), microwave, ultrasonic and light can be transferred into the cornea. Non-thermal energy does not include the concept of heating a tip that had been inserted or is to be inserted into the cornea.

By way of example, the controller can be modified to supply energy in the microwave frequency range or mechanical-acoustical energy in the ultrasonic frequency range. By way of example, the probe may have a helical microwave antenna with a diameter suitable for corneal delivery. The delivery of microwave energy could be achieved with or without corneal penetration, depending on the design of the antenna. The system may modulate the microwave energy in response to changes in the characteristic impedance.

For ultrasonic application, the probe would contain a transducer that is driven by the controller and mechanically oscillates a tip of the probe. The system could monitor acoustic impedance and provide a corresponding feedback/regulation scheme. For application of photonic energy the probe may contain some type of light guide that is focused on and/or inserted into the cornea and directs photonic energy into corneal tissue. The controller would have means to generate photonic energy, preferably a coherent light source such as a laser or a flash tube such as xenon, that can be delivered by the probe. The probe may include lens, waveguide and a phototransducer that is used sense reflected photonic energy and monitor variations in the index of refraction, birefringence index of the cornea tissue as a way to monitor physiological changes and regulate power.

What is claimed is:

1. An apparatus that is used in a medical procedure on a cornea, comprising:
a probe that delivers energy; and,
a mechanism that moves said probe about the cornea.

2. The apparatus of claim 1, wherein said mechanism includes a ring that is placed onto the cornea, a block that supports said probe and a first actuator that moves said block about said ring.

3. The apparatus of claim 2, wherein said mechanism includes a second actuator that moves said probe into contact with the cornea.

4. The apparatus of claim 3, wherein said mechanism includes a third actuator that moves said probe to different radial locations on the cornea.

5. The apparatus of claim 4, wherein the radial locations are 2 to 5 millimeters from a center of the cornea.

6. The apparatus of claim 1, wherein said probe delivers energy to denature corneal tissue.

7. The apparatus of claim 1, further comprising a controller that controls said mechanism.

8. The apparatus of claim 7, wherein said probe delivers energy to denature corneal tissue and said controller moves said probe to create a circular pattern of denatured areas.

9. An apparatus that is used in a medical procedure on a cornea, comprising:
probe means for delivering energy; and,
mechanism means for moving said probe about the cornea.

10. The apparatus of claim 9, wherein said mechanism means includes a ring that is placed onto the cornea, a block that supports said probe and a first actuator that moves said block about said ring.

11. The apparatus of claim 10, wherein said mechanism means includes a second actuator that moves said probe means into contact with the cornea.

12. The apparatus of claim 11, wherein said mechanism means includes a third actuator that moves said probe means to different radial locations on the cornea.

13. The apparatus of claim 12, wherein the radial locations are 2 to 5 millimeters from a center of the cornea.

14. The apparatus of claim 9, wherein said probe means delivers energy to denature corneal tissue.

15. The apparatus of claim 9, further comprising a controller that controls said mechanism means.

16. The apparatus of claim 8, wherein said probe means delivers energy to denature corneal tissue and said controller moves said probe means to create a circular pattern of denatured areas.

17. A method for performing a medical procedure on a cornea, comprising:
automatically moving a probe into contact with a cornea;
delivering energy to the cornea through the probe to denature corneal tissue; and,
automatically moving the probe to a new location of the cornea.

18. The method of claim 17, wherein the probe is moved about the cornea and delivers energy to create a pattern of denatured areas in the cornea.

19. The method of claim 18, further comprising automatically moving the probe to different radial positions on the cornea.

20. The method of claim 19, wherein the radial positions are 2 to 5 millimeters from a center of the cornea.

21. An ophthalmic ring assembly, comprising:
a ring that can be placed onto the cornea;
a block coupled to said ring; and,
a first actuator that moves said block about said ring.

22. The apparatus of claim 21, further comprising a second actuator structurally coupled to said ring.

23. The apparatus of claim 22, further comprising a third actuator coupled to said block.

24. The apparatus of claim 21, further comprising a controller coupled to said first actuator.

25. The apparatus of claim 23, further comprising a controller coupled to said first, second and third actuators.
